(19) United States
(12) Patent Application Publication
(10) Pub. No.: US 2021/0060058 A1
(43) Pub. Date: Mar. 4, 2021

(54) COMPOSITIONS AND METHODS FOR TREATING HYDROGEN SULFIDE-MEDIATED HYPERDYNAMIC CIRCULATION

(71) Applicants: UNM Rainforest Innovations, Albuquerque, NM (US); Veterans Affairs, Washington, DC (US)

(72) Inventors: Aleksandr Birg, Albuquerque, NM (US); Henry C. Lin, Albuquerque, NM (US); Nancy L. Kanagy, Albuquerque, NM (US)

(21) Appl. No.: 17/012,871

(22) Filed: Sep. 4, 2020

Related U.S. Application Data
(60) Provisional application No. 62/895,692, filed on Sep. 4, 2019.

(51) Int. Cl. A61K 33/08; A61K 33/245; A61K 33/30; G01N 33/497; G01N 33/493

(52) U.S. Cl. CPC A61K 33/08; A61K 33/245; G01N 33/493; G01N 33/497; A61K 33/30

(57) ABSTRACT

A pharmaceutical composition generally includes a compound that reduces H₂S in the gut of a subject and a pharmaceutically acceptable carrier. In some embodiments, the compound that reduces H₂S in the gut of a subject can include magnesium or a salt thereof, bismuth or a salt thereof, molybdenum or a salt thereof, zinc or a salt thereof, iron or a salt thereof, nickel or a salt thereof, nitrate or a salt thereof, cobinamide or a salt thereof, a heme protein, an antimicrobial, an antibiotic, a prebiotic, a probiotic, or a synbiotic.

In another aspect, a method of detecting H₂S-mediated hyperdynamic circulation, or a consequence thereof in a subject having, or at risk of having, H₂S-mediated hyperdynamic circulation generally includes obtaining a biological sample from the subject, measuring H₂S in the sample, and identifying the subject as having H₂S-mediated hyperdynamic circulation if the H₂S measured in the sample is greater than a predetermined threshold. In some embodiments, the biological sample can be plasma or blood. In some of these embodiments, the predetermined threshold is a plasma H₂S concentration of 45 μM. In other embodiments, the predetermined threshold is a baseline plasma H₂S concentration measured from a previous sample from the subject.

### CROSS-REFERENCE TO RELATED APPLICATION

This application claims the benefit of U.S. Provisional Patent Application No. 62/895,692, filed Sep. 4, 2019, which is incorporated herein by reference in its entirety.

### GOVERNMENT FUNDING

This invention was made with government support under HL123301 awarded by the National Institutes of Health. The government has certain rights in the invention.

### SUMMARY

This disclosure describes, in one aspect, a pharmaceutical composition that generally includes a compound that reduces hydrogen sulfide (H₂S) in the gut of a subject and a pharmaceutically acceptable carrier.

In some embodiments, the compound that reduces H₂S in the gut of a subject can include magnesium or a salt thereof, bismuth or a salt thereof, molybdenum or a salt thereof, zinc or a salt thereof, iron or a salt thereof, nickel or a salt thereof, nitrate or a salt thereof, cobinamide or a salt thereof, a heme protein, an antimicrobial, an antibiotic, a prebiotic, a probiotic, or a synbiotic.

In another aspect, this disclosure describes a method of detecting H₂S-mediated hyperdynamic circulation, or a consequence thereof, in a subject having, or at risk of having, H₂S-mediated hyperdynamic circulation. Generally, the method includes obtaining a biological sample from the subject, measuring H₂S in the sample, and identifying the subject as having H₂S-mediated hyperdynamic circulation if the H₂S measured in the sample is greater than a predetermined threshold.

In some embodiments, the biological sample can be plasma or blood. In some of these embodiments, the predetermined threshold is a plasma H₂S concentration of 45 μM. In other embodiments, the predetermined threshold is a baseline plasma H₂S concentration measured from a previous sample from the subject.

In some embodiments, the biological sample is a breath. In some of these embodiments, the predetermined threshold is a baseline breath H₂S concentration measured from a previous sample from the subject.

In another aspect, this disclosure describes a method of detecting H₂S-mediated hyperdynamic circulation, or a consequence thereof, in a subject having, or at risk of having, H₂S-mediated hyperdynamic circulation. Generally, the method includes obtaining a biological sample from the subject, measuring thiosulfate in the sample, and identifying the subject as having H₂S-mediated hyperdynamic circulation if the thiosulfate measured in the sample is greater than a predetermined threshold.

In some embodiments, the biological sample is plasma or blood. In some of these embodiments, the predetermined threshold is a plasma thiosulfate concentration of 1.13 mg/dl. In other embodiments, the predetermined threshold is a baseline plasma thiosulfate concentration measured from a previous sample from the subject.

In some embodiments, the biological sample is urine. In some of these embodiments, the predetermined threshold is a urine thiosulfate concentration of 0.28 mg/dl. In other embodiments, the predetermined threshold is a baseline urine thiosulfate concentration measured from a previous sample from the subject.

In another aspect, this disclosure describes a method of treating a subject having, or at risk of having, H₂S-mediated hyperdynamic circulation, or a consequence of H₂S-mediated hyperdynamic circulation. Generally, the method includes administering to the subject a composition that includes a compound that reduces H₂S in the gut of the subject in an amount effective to treat H₂S-mediated hyperdynamic circulation or the consequence of H₂S-mediated hyperdynamic circulation.

In some embodiments, the compound that reduces H₂S in the gut of a subject can include magnesium or a salt thereof, bismuth or a salt thereof, molybdenum or a salt thereof, zinc or a salt thereof, iron or a salt thereof, nickel or a salt thereof, nitrate or a salt thereof, cobinamide or a salt thereof, a heme protein, an antimicrobial, an antibiotic, a prebiotic, a probiotic, or a synbiotic.

In some embodiments, the treatment is prophylactic. In other embodiments, the treatment is therapeutic.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Portal venous flow (mL/min) recordings over 30-minute jejunal infusion of NaHS in four groups: Standard-chow-fed rats (Standard), High-fat diet (HFD), High-fat diet combined with bismuth subsalicylate (HFD+Bismuth), and standard chow combined with bismuth subsalicylate (STD+Bis). Initial baseline flow measurements recorded and averaged (shown as Time 0) for each group. All measurements recorded as average raw flow rates±SEM. **statistically significant difference between HFD group and three other groups with arrow indicating the first time interval where a statistically significant difference is noted.

FIG. 2. Systemic venous pressure (mmHg) recordings over 30-minute jejunal infusion of NaHS in four groups: Standard-chow-fed rats (Standard), High-fat diet (HFD), High-fat diet combined with bismuth subsalicylate (HFD+Bismuth) and standard chow combined with bismuth subsalicylate (STD+Bis). Initial baseline pressures recorded and averaged (shown as Time 0) for each group. All measurements recorded as average raw values±SEM. No statistical difference is seen between the four groups throughout infusion.

FIG. 3. Measurement of systemic levels of aspartate transaminase (AST), units per liter, in all four diet groups. Levels obtained at end of 10-week diet. All measurements represent mean±95% confidence interval. ***statistically significant difference between AST measurements in HFD group and the three other groups.

FIG. 4. Measurement of systemic levels of alanine transaminase (ALT), units per liter, in all four diet groups. Levels obtained at end of 10-week diet. All measurements represent mean±95% confidence interval. No statistical difference seen between the four groups.

FIG. 5. Measurement of systemic levels of total bilirubin (Tbili), milligrams per deciliter (mg/dL), in all four diet groups. Levels obtained at end of 10-week diet. All measurements represent mean±95% confidence interval. No statistical difference seen between the four groups.

FIG. 6. Measurement of systemic levels of albumin (Alb), grams per deciliter (g/dL), in all four diet groups. Levels obtained at end of 10-week diet. All measurements represent mean±95% confidence interval. No statistical difference seen between the four groups.

FIG. 7. Portal venous flow recordings over 20-minute infusion of NaHS in two groups: Standard-diet-fed rats and Magnesium Oxide (MgO)-fed rats. Vehicle group shown as control; standard diet fed rats followed by water infusion. All measurements shown as percent change from baseline to accommodate for compensate for variation in baseline data; data shown as percent change±SEM. Baseline flow measurements recorded and average (shown as time 0) for each group. Infusion started at minute 1. **statistically significant different between MgO diet group and standard diet group. No difference is noted between MgO diet group and vehicle.

FIG. 8. Effect of molybdate on growth of Desulfovibrio vulgaris (DSV) on a culture plate. In a dose-dependent fashion, molybdate (molb) at 10 μM, 50 μM, and 1 mM suppressed the growth of DSV over a period of 14 hours with molybdate at 1 mM completely suppressing the growth of this sulfate reducing bacteria.

FIG. 9. Effect of molybdate on the production of hydrogen sulfide gas measured in parts per billion (ppb) in the culture system. In a dose dependent fashion, molybdate (molb) at 10 μM, 50 μM, and 1 mM suppressed the generation of H₂S gas by DSV. These results support the potential use of molybdate as a treatment for conditions associated with exposure to excessive H₂S.

### DETAILED DESCRIPTION OF ILLUSTRATIVE EMBODIMENTS

This disclosure describes compositions and methods for treating vascular complications of portal hypertension in the setting of liver disease. Generally, the compositions include a compound that reduces H₂S in the gut (e.g., bismuth subsalicylate). Decompensations in liver disease are known to have high mortality and include severe bleeding, infections, kidney disease, and pulmonary disease. This disclosure describes compounds that reduce H₂S in the gut as a prophylactic and/or therapeutic treatment to reduce complications of liver disease and possible use in treatment of previously developed complications.

A composition that includes a compound that reduces H₂S in the gut (e.g., bismuth subsalicylate, magnesium, etc.) may therefore be used to treat, for example, variceal formation (e.g., esophageal varices, gastric varices, intestinal varices, rectal varices, etc.), variceal bleeding, bleeding from portal hypertensive gastropathy, portal hypertensive enteropathy, portal hypertensive colonopathy, other gastrointestinal bleeding, ascites, hepatic cardiac syndrome, cirrhotic cardiomyopathy, heart failure, hypervolemic hyponatremia, electrolyte imbalances, renal failure, sodium retention, fluid retention, tissue edema, pleural effusion, hypoxia, hypoxemia, hepatorenal syndrome, portopulmonary syndrome, hepatopulmonary syndrome, portopulmonary hypertension, jaundice, splenomegaly, spontaneous bacterial peritonitis, porto-systemic anastomosis formation, impaired detoxification, bacterial translocation, liver failure, fibrosis formation, cirrhosis formation and its complications, brain swelling, or hepatic encephalopathy, or coma.

Treatment can be administered prophylactically or as a therapeutic treatment to limit the severity and/or the extent of symptoms or clinical signs of liver disease once complications occur.

Abnormal blood flow is common in liver diseases. Specifically, a pattern of blood flow termed “hyperdynamic circulation” is associated with cirrhosis and is characterized by vasodilation, low systemic blood pressure, greater than normal cardiac output, and/or abnormally elevated regional blood flow to abdominal organs. Increased blood flow to the splanchnic circulation involving the celiac trunk, the superior mesenteric artery, and the inferior mesenteric artery—which serve the stomach, small intestine, large intestine, pancreas, liver, spleen, and kidneys—is responsible for portal hypertension with severity of this syndrome driven by increased blood flow through the portal circulatory system.

The compensatory response of the vasculature to portal hypertension includes the development of porto-systemic collaterals such as esophageal, gastric, intestinal, and/or rectal varices. Increased blood flow also drives mucosal changes such as, for example, portal hypertensive gastropathy, enteropathy, and/or colonopathy. The abnormal diversion of blood away from the liver can lead to liver failure and/or encephalopathy as evidenced by toxins from the gut bypassing the detoxifying actions of hepatic metabolism. Increased splanchnic blood flow serves as an ongoing driver for further expansion and dilation of collaterals, which can lead to complications such as, for example, variceal bleeding.

The increase in blood flow seen in hyperdynamic circulation upregulates the expression of nitric oxide, which can lead to vasodilation. Vasodilation, in turn, lowers the systemic blood pressure and can create a state of relative volume depletion that drives the sympathetic nervous system, the release of vasopressin/antidiuretic hormone (ADH) in the absence of a rise in plasma osmolality, and/or the activation of the renin-angiotensin-aldosterone system, which can affect homeostasis of renal management of fluids and electrolytes. The adverse effects include, for example, hypervolemic hyponatremia despite abnormal sodium retention, abnormal free water retention, and increased total plasma volume. Impaired glomerular filtration rates and renal failure can follow as the compensatory mechanisms of the kidney fail to balance the multiple vasodilators and vasoconstrictors in play. Hepato-renal syndrome is one consequence of this adverse effect on renal function. Renal dysfunction further promotes the formation of ascites or pleural effusion driven initially by abnormal sodium and water retention. Vasodilation of cerebral capillaries and increased blood flow contribute to encephalopathy, brain edema, and even coma by promoting the diffusion of toxins that originate from the gut (e.g., ammonia) across the blood-brain barrier and by increasing the direction of flow from inside to outside the vessels. Vasodilation contributes to hypoxemia/hypoxia by increasing mixing of well-oxygenated blood and poorly-oxygenated blood in the peripheral parts of the circulation at the level of capillaries and venules that is the hallmark of hepato-pulmonary syndrome. With vasodilation driven by abnormal blood flow, the normal vasoconstriction of pulmonary regional vascular beds in response to hypoxia can be impaired. This can result in hypoxemia due to a V/Q mismatch. Abnormal blood flow leads to remodeling of blood vessels with pulmonary hypertension as a potential complication. To compensate for the vasodilation due to increased blood flow, cardiac output and heart rate are often increased in cirrhosis. When the effect is prolonged, cardiac contractile dysfunction can ensue, driven at least in part by the pro-inflammatory effects of a high output state leading to cardiac or heart failure/syndrome.

With the cause of the high blood flow not understood, the various adverse clinical consequences of hyperdynamic circulation-portal hypertension, varices and their complication of bleeding, hypoxemia, hepatopulmonary syndrome, renal dysfunctions, sodium retention, renal failure, ascites formation, encephalopathy, brain swelling, coma, and heart failure, are treated currently in a piece-meal fashion. For example, bands are placed on esophageal varices to stop or prevent variceal bleeding or a diuretic is administered to a patient with ascites. Understanding the cause of the increased blood flow could lead to novel diagnostic and therapeutic interventions in advanced liver disease and other conditions driven by similar pathophysiology.

Hydrogen sulfide (H₂S) is a gaso-transmitter that is a mediator of the vascular system. Endogenous H₂S produced by a host regulates blood pressure via its effects on the vascular system, but the effect of gut-bacteria-derived H₂S, a by-product of fermentation by sulfate reducing bacteria (SRB), is mostly unknown. The healthy gut microbiome, mostly confined to the colon, includes sulfate reducing bacteria, but the generated H₂S is detoxified by endogenous enzymes in the colon. An increase in SRB population in the gut microbiome has been proposed in the setting of dysbiosis (abnormal and deleterious changes to the microbiome). An overgrowth of sulfate reducing bacteria can colonize the small intestine, a more proximal section of the intestine that does not have the same detoxifying mechanism found in the colon. This overgrowth of the sulfate reducing bacteria population can increase H₂S production in the gut lumen, the absorption of the gas across the intestinal lining, and entry into the circulation with deleterious effects on the vasculature. Once absorbed from the intestine, if not completely detoxified, H₂S enters the blood vessels that drain the intestine (mesenteric veins) and the portal vein before entering the liver via the hepatic sinusoids. If not completely detoxified in the liver, the gas exits the liver and joins the systemic circulation, where the H₂S can access all organs. As H₂S concentration can be measured in both arterial and venous blood, the effects of H₂S on organs such as the kidneys are not limited to its delivery by arterial blood alone. The passage of H₂S from the intestinal lumen to the systemic circulation (which normally occurs from the mesenteric circulation to the portal circulation to hepatic sinusoids to hepatic veins to the inferior vena cava and, finally, to the systemic circulation) may be shortened if normal porto-systemic shunts exist such as rectal varices or esophageal varices that would allow H₂S to enter the systemic circulation from the mesenteric/portal circulation without going through the liver.

Patients with underlying liver disease, including fatty liver disease, have a higher likelihood of having dysbiosis. Obesity can contribute to alterations of the microbiome that lead to dysbiosis and can be a factor in developing non-alcoholic fatty liver disease (NAFLD). Dysbiotic alterations in the microbiome have also been implicated in the development of NAFLD independent of obesity.

H₂S contributes to the dysregulation of the portal vasculature in the setting of liver disease. However, the role of gut bacteria-derived H₂S has not been considered. Indeed, overproduction of gut-derived H₂S in the setting of dysbiosis in patients with liver disease may uniquely affect the portal circulation, contributing to vascular complications of portal hypertension. Therefore, in patients with liver disease with elevated numbers of sulfate reducing bacteria, expansion of the bacterial colonization to include the small intestine—e.g., small intestinal bacterial overgrowth—can lead to increased absorption of H₂S into the mesenteric/portal circulation. Increased concentration of bacteria-derived H₂S in blood can negatively influence the portal circulation.

This disclosure therefore describes treatment directed at H₂S or H₂S-generating bacteria to reverse the increased blood flow so as to treat the consequences of hyperdynamic circulation. Compounds that reduce intestinal H₂S (e.g., bismuth subsalicylate, BSS) can reverse intestinal dysfunction after H₂S exposure. This disclosure describes the use of compounds that reduce intestinal H₂S as a therapeutic and/or prophylactic agent for treating H₂S-mediated complications of liver disease.

This disclosure further describes methods of detecting H₂S-mediated complications of liver disease. Bacteria-derived H₂S may enter the circulation and present to the pulmonary vascular bed so that the gas appears in the exhaled breath. Measuring the concentration of H₂S in the blood or plasma, urine, and the exhaled breath can detect the H₂S-mediated mechanism underlying hyperdynamic circulation. Monitoring the concentration of H₂S may help to manage the patient by identifying patients exposed to abnormally high concentrations of H₂S and assessing the adequacy of treatment directed at reducing the H₂S exposure.

Rats were fed a high fat diet (HFD) to induce dysbiosis and fatty liver disease. Responses in control group rats (fed standard chow) were compared to responses in three experimental groups: HFD diet, HFD+BSS, and normal chow+BSS. After eight weeks, all rats returned to normal chow, but the BSS groups continued to receive BSS in the normal chow. After 10 weeks, systemic blood was collected for measurements of liver function tests (AST, ALT, Tbili, Alb) before further experimentation. After blood collection, rats underwent surgery to measure porta vein flow and systemic venous blood pressure before and after infusion of small intestine (jejunum) sodium hydrosulfide (NaHS), a hydrogen sulfide donor.

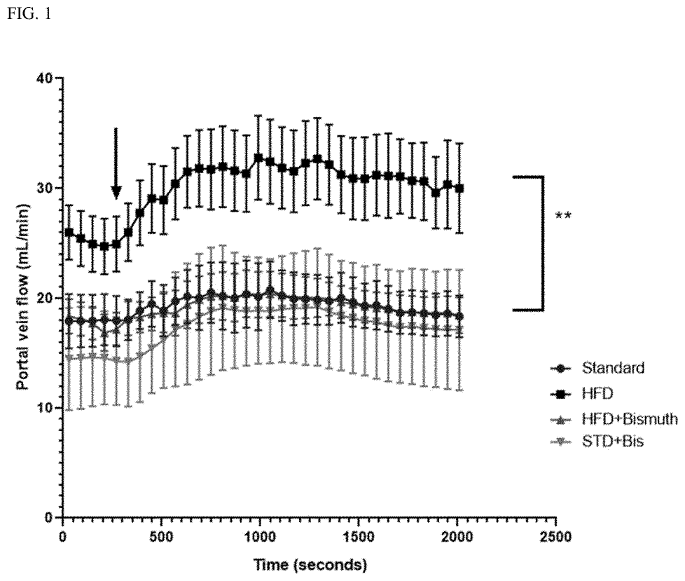

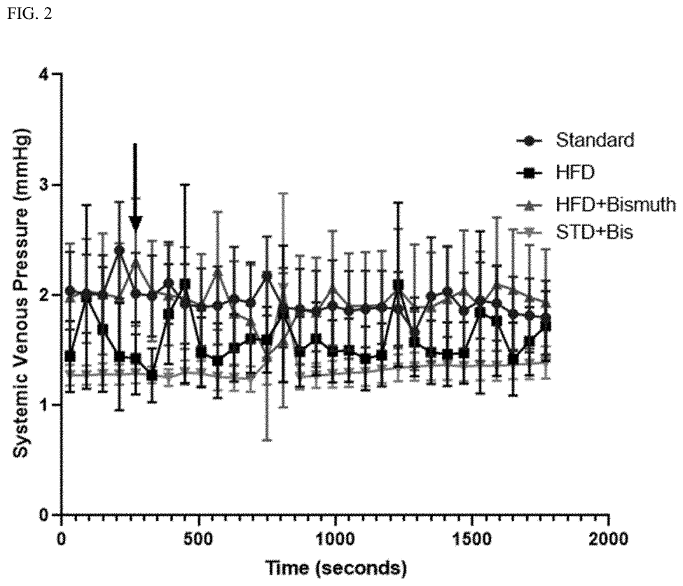

Portal vein blood flow was significant higher at baseline [24.9±2.5] and throughout the study in the HFD group compared to rats given HFD+BSS [17.2±1.6], standard diet+BSS [14.3±2], or standard chow [17.9±2.3] (FIG. 1). HFD+BSS was not different at baseline or during NaHS infusion compared to standard chow or standard chow+BSS groups. Systemic venous pressure was not different between the four groups at any time: HFD [1.4±0.3], HFD+BSS [2.3±0.6], standard chow+BSS [1.3±0.1], and standard chow [2.0±0.4] (FIG. 2).

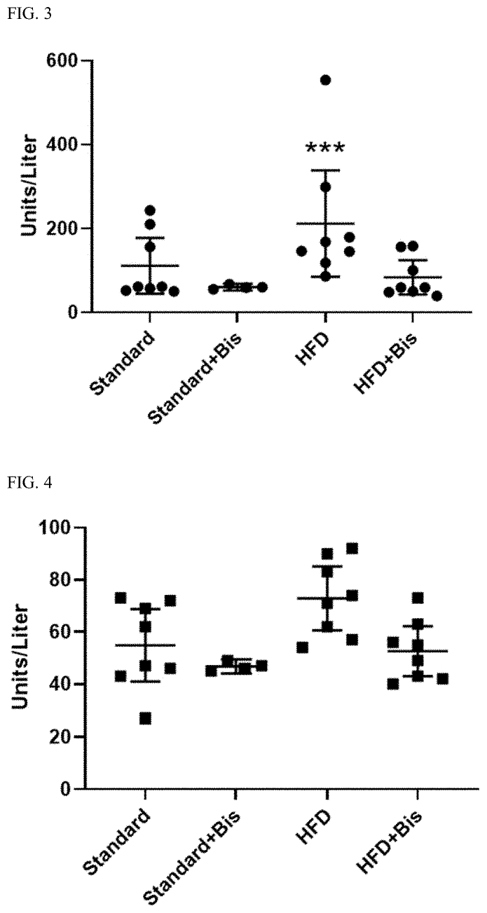

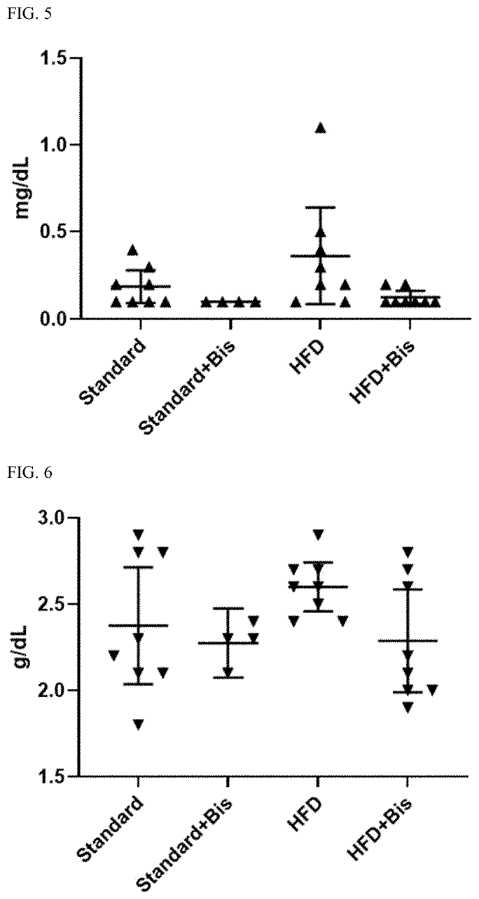

There was a statistically significant rise in AST in high fat diet group compared to other diets (FIG. 3). The rise in AST alone is not indicative of developing fatty liver disease in HFD group. However, the ratio of AST/ALT being greater than 1 is indicative of developing fibrosis. Albumin levels (FIG. 6) shows that the levels remain within normal range for Sprague-Dawley rats, suggesting that these rats did not develop liver synthetic dysfunction.

These data show that H₂S within the jejunum increases portal vein blood flow that is augmented with concurrent HFD-induced fatty liver disease. The effect of H₂S on portal vein flow is suppressed with BSS delivered in the food to eliminate HFD-augmented sensitivity of the portal vein to H₂S.

Thus, this disclosure describes compositions and methods that are useful for diagnosing, treating, and/or reducing hyperdynamic circulation—and its consequences—in patients with liver disease. In some embodiments, the hyperdynamic circulation and/or its consequences may be associated with gut dysbiosis accompanied by increased production and exposure to H₂S.

In one aspect, hyperdynamic circulation and/or its consequences may be diagnosed by a blood or urine test measuring the concentration of hydrogen sulfide or its metabolites or by a breath test showing a high concentration of H₂S. For example, H₂S concentration can be tested via assay of blood or plasma sample or measured in exhaled breath via breath testing. Thiosulfate, a metabolite of H₂S may be measured in blood or plasma or urine. A breath test may be performed by fasting the subject before administering a fermentable substrate (e.g., lactulose, glucose, etc.) and then taking samples of exhaled breath at regular intervals for a period of time. The breath samples are then measured in commercial gas chromatographs to measure concentrations of hydrogen (H₂) and methane (CH₄) while carbon dioxide concentration is used to normalize the data. H₂S can also be measured in the exhaled breath using the same breath gas sample. These gases may be measured separately or all together.

H₂S may be measured using any suitable method. In one exemplary embodiment, H₂S can be measured in the blood as previously described (Kevil et al., Methods Enzymol. 2015, 554:31-45; Suguhara et al., Anal Sci, 2016, 32:1129-1131). Briefly, the monobromobimane (MBB) method is coupled with reversed phase high-performance liquid chromatography (RP-HPLC) and fluorescence detection to measure nM concentrations of H₂S. The method involves the derivatization of sulfide with excess MBB under precise reaction conditions at room temperature in a low 02 environment to form sulfide dibimane (SDB). The resultant fluorescent SDB is analyzed by RP-HPLC using fluorescence detection with the limit of detection for SDB (2 nM). Rapidly processing the blood sample provides more accurate readings by reducing loss of the dissolved H₂S. Other approaches to measuring H₂S include but are not limited to methylene blue test, absorbance-based methods, other chromatographic methods, electrochemical techniques, or fluorometric detection methods.

An exemplary methylene blue test is described in Moest, R. R., 1975, Anal. Chem. 47:1204-4205.

Exemplary absorbance-based methods are described, for example, in Ni et al., 2015, Sensors Actuators, B Chem. 220:210-21:5; Ahn et al., 2017, Spectrochim. Acta—Part A Mol. Biomol. Spectrosc. 177:118-124; and Jarosz et al., 2013. Anal. Chem. 85:3638-3643.

Exemplary alternative chromatographic methods are described in, for example, Rath et al., 1980, Chromatographia 13:513-514 and Heshka et al., 2014, J. Sep. Sci. 37:3649-3655.

Exemplary electrochemical techniques are described in, for example, Baumann, E. W., 1974, Anal. Chem. 46:1345-1347; Flu et al., 2013, Rev. Anal. Chem. 32:247-256; Kolluru et al., 2013, Nitric Oxide—Biol. Chem. 35:5-20; Jeroschewski et al., 1996, Anal. Chem. 68:4351-4357; Jeroschewski et al., 1993, Fresenius. J. Anal. Chem. 346:930-933; and Hall et al., 2018, Anal. Chem. 90:M94-5200),

Exemplary fluorometric detection methods are described in, for example, Chemistry, Biochemistry and Pharmacology of Hydrogen Sulfide. (Springer, 2015, Moore, P K and Whiteman, M eds,), Lin et al., 2015, Chem. Soc. Rev. 44:4596-4618; Karunya et al., 2019, Scientific Reports 9: 3258).

Thiosulfate, the metabolite of H₂S, could be measured using liquid chromatography as described in, for example, Shea et al., 1984, Anal Biochem 140(2):589-594; Hildebrandt et al., 2008 FEBS J 275(13):3352-3361, and Kage et al., 1991 J Analytical Toxicology 15(3):148-150.

The normal concentration of plasma H₂S is around 45 μM. An increase in H₂S by 5 μM or more is considered “high” and is cause for further testing for excess production. In some cases, the threshold increase in plasma H₂S concentration to be considered “high” is an increase by at least 10 μM. In some embodiments, the baseline against which an increase in H₂S is measured the “normal” standard of 45 μM. In other embodiments, the baseline against which the plasma H₂S concentration is compared may be the H₂S concentration from a previous sample taken from the subject.

Normal concentration of plasma thiosulfate is 1.13±0.11 mg/dl. The normal concentration of urine thiosulfate is 0.28±0.02 mg/dl. An increase of 0.25 mg/dl in plasma thiosulfate or an increase of 0.08 mg/dl in urine thiosulfate is considered “high” and is cause for further testing. In some embodiments, the baseline against which an increase in plasma thiosulfate or urine thiosulfate is compared is the normal standard provided above. In other embodiments, the baseline against which the plasma thiosulfate or urine thiosulfate is compared is the plasma thiosulfate or urine thiosulfate concentration from a previous sample taken from the subject.

In another aspect, hyperdynamic circulation and its consequences may be treated by administering to a subject a composition that includes a compound that reduces H₂S in the gut or reduces the number of sulfate reducing bacteria. As used herein, the term “treat” or variations thereof refer to reducing, limiting progression, ameliorating, or resolving, to any extent, symptoms and/or clinical signs related to a condition. As used herein, the term “symptom” refers to any subjective evidence of disease or of a patient's condition, while the term “sign” or “clinical sign” refers to an objective physical finding relating to a particular condition capable of being found by one other than the patient.

As explained in more detail below, “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject.

Consequences of hyperdynamic circulation that may be treated using the compositions and methods described herein include, but are not limited to, portal hypertension, varices formation (e.g., esophageal varices, gastric varices, intestinal varices, rectal varices, etc.), variceal bleeding, portal hypertensive gastropathy, gastric antral vascular ectasia, portal hypertensive enteropathy, portal hypertensive colonopathy, liver failure, hepatic encephalopathy, coma, variceal bleeding, hypervolemic hyponatremia, electrolyte imbalances, renal failure, sodium retention, fluid retention, tissue edema, portopulmonary syndrome, hepatopulmonary syndrome, pulmonary hypertension, jaundice, splenomegaly, portosystemic anastomosis formation, impaired hepatic detoxification, bacterial translocation, liver failure, fibrosis formation cirrhosis and its complications, brain swelling, hepatorenal syndrome, ascites, spontaneous bacterial peritonitis, pleural effusion, hypoxemia, hypoxia, hepato-pulmonary syndrome, porto-pulmonary syndrome, hepatic cardiac syndrome, cirrhotic cardiomyopathy, or heart failure.

Symptoms and clinical signs of hyperdynamic circulation and the consequences thereof include, but are not limited to, hematemesis, coffee-ground emesis, hematochezia, passing blood from the rectum, passing melena from the rectum, easy bruising, nausea, vomiting, early satiety, gastroesophageal reflux, dysphagia, anorexia, bloating, excessive gas, excessive flatus, jaundice, confusion, altered mental status, difficulty with concentration, impaired memory, insomnia, asterixis, fogginess of head, edema, abdominal swelling, abdominal pain, difficulty with breathing, shortness of breath, fatigue, change in weight, chest pain, palpitations of the heart, decreased urine output, leg edema or swelling, or full body swelling (anasarca).

In some embodiments, the conditions, symptoms, and/or clinical signs may be related to obesity, fatty liver disease, metabolic syndrome, alcoholic liver disease, steatosis, steatohepatitis, hepatitis, cirrhosis, other acute or chronic liver disease, heart failure, respiratory failure, or kidney failure.

In some embodiments, the conditions, symptoms, and/or clinical signs may be related to bacterial overgrowth or other gut dysbiosis. Gut dysbiosis could represent an abnormal expansion of the gut microbial community, small or large intestinal bacterial overgrowth, abnormal increase in the microbial density, an abnormal composition of the gut microbial community or an abnormal pattern of fermentation or generation of H₂S, or other gaseous byproducts of fermentation.

Exemplary compounds that reduce H₂S in the gut include, but are not limited to, bismuth subsalicylate, bismuth citrate, bismuth(III) deferiprone, other bismuth salts, molybdate, magnesium oxide, magnesium citrate, magnesium glycinate, magnesium lysinate chelate, magnesium chloride, magnesium glycerophosphage, magnesium hydroxide, magnesium carbonate, magnesium orotate dehydrate, magnesium sulfate, magnesium peroxide or other oxygen emitters, any other magnesium salt, iron (e.g., ferric chloride, other ferric iron, ferrous iron, etc.) zinc oxide or other zinc salt, metal salts, heavy metals, nitrates, nitrites, nitrous acid, cobinamide, a heme protein, human serum proteins, an antimicrobial, an antibiotic (including poorly absorbable antibiotic such as rifaximin, e.g., an antibiotic that specifically target sulfate-reducing bacteria), a prebiotic, a probiotic, or a synbiotic. Additional metal salt compounds include salts of copper, nickel, iron, manganese, cadmium, or chromium.

In some embodiments, the compound can be poorly absorbed and therefore presents low serum availability but high availability in the intestinal lumen. Further, in some embodiments, the compound can possess a release profile and/or gastrointestinal transit profile that allows the compound to be maximally available in the small intestine during the time window when bacterial fermentation takes place following a meal when hydrogen gas is generated since sulfate reducing bacteria converts hydrogen to hydrogen sulfide by means of dissimilatory sulfate reduction or by fermentative bacteria that metabolize organic sulfur compounds. Bismuth subsalicylate, cobinamide, and heme proteins bind the gas, reducing its ability to adversely affect the patient. Molybdate blocks the H₂S production pathway of sulfate reducing bacteria, therefore reducing the gas production itself. The presence of magnesium inhibits the activity of ATP sulfurylase, the first of three enzymes in the sulfate reduction pathway. Magnesium peroxide, magnesium hydroxide, other magnesium salts or zinc sulfide inhibit sulfate reducing bacteria. Antibiotics, antimicrobials, prebiotics, probiotics, and synbiotics can reduce the total population of H₂S-producing microbes in the gut and, therefore, and limit the total potential H₂S production.

The compound that reduces H₂S in the gut, or a combination of such compounds, may be formulated into a pharmaceutical composition by combining the active agent with a pharmaceutically acceptable carrier. As used herein, the term active agent refers collectively to either a single compound that reduces H₂S in the gut or a combination of such compounds; thus, noun-verb agreement for the term “active agent” should not be construed as requiring a single compound that reduces H₂S in the gut.

As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the active agent without causing any undesirable biological effects or interacting in a deleterious manner with any of the other components of the pharmaceutical composition in which it is contained.

The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), rectal, or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol), or oral mucosa via disintegrating/dissolving route. A composition also can be administered via a sustained or delayed release. In some embodiments, the composition can be formulated to optimize the time of contact of the active agent with H₂S—generating sulfate reducing bacteria during the time period of microbial fermentation after a meal.

Thus, the active agent may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the active agent into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The method includes administering an effective amount of the composition to a subject having, or at risk of having, a particular condition. In this aspect, an “effective amount” is an amount effective to reduce, limit progression, ameliorate, or resolve, to any extent, a symptom or clinical sign related to the condition.

The amount of active agent administered can vary depending on various factors including, but not limited to, the specific active agent being administered, the weight, physical condition, and/or age of the subject, and/or the route of administration. Thus, the absolute weight of the active agent included in a given unit dosage form can vary widely, and depends upon factors such as the species, age, weight and physical condition of the subject, and/or the method of administration. Accordingly, it is not practical to set forth generally the amount that constitutes an amount of the active agent effective for all possible applications. Those of ordinary skill in the art, however, can readily determine the appropriate amount with due consideration of such factors.

For example, certain active agents may be administered at the same dose and frequency for which the active agent has received regulatory approval. In other cases, certain active agents may be administered at the same dose and frequency at which the active agent is being evaluated in clinical or preclinical studies. One can alter the dosages and/or frequency as needed to achieve a desired level effect from the active agent. Thus, one can use standard/known dosing regimens and/or customize dosing as needed.

The active agents act in the gut lumen, where hydrogen sulfide is available. Thus, gut luminal concentrations are more relevant than blood concentrations. Thus, an effective amount of active agent may be significantly less than reported effective doses of the active agent for other indications since the active agents act directly in the gut rather than being absorbed and diluted in the plasma. Multiple metal compounds can be used in organic and inorganic forms (e.g., magnesium oxide, magnesium citrate) and different bioavailabilities are noted based on the effect of the different formulations on blood concentrations. Since the active agents act in the gut lumen, prior to any physiologic absorption and utilization, the effective concentrations may be lower than listed above, and lower than doses required to produce similar plasma-based bioavailability as reported in literature.

The daily dose of some active agents is provided as the mass of active agent (mg/day). For other active agents, the daily dose is expressed in terms of the mass of active agent relative to the mass of the subject (mg/kg/day). The general dosage amounts that follow are, for brevity, provided only in terms of the daily mass of active agent. Unless specified for a particular active agent, the values provided below for the mass of active agent in a minimum daily dose, a maximum daily dose, or the endpoints for a range of daily dose values are applicable values for daily doses in terms of mass (mg/day) or the mass of active agent relative to the mass of the subject (mg/kg/day), as may be appropriate for a selected active agent.

In some embodiments, the method can include administering sufficient active agent to provide a minimum daily dose (mg/day) of at least 0.001 mg such as, for example, at least 0.0012 mg, at least 0.0024 mg, at least 0.5 mg, at least 1 mg, at least 1.5 mg, at least 2 mg, at least 3 mg, at least 4 mg, at least 5 mg, at least 6 mg, at least 7 mg, at least 8 mg, at least 9 mg, at least 10 mg, at least 15 mg, at least 20 mg, at least 30 mg, at least 33 mg, at least 35 mg, at least 40 mg, at least 45 mg, at least 50 mg, at least 60 mg, at least 70 mg, at least 80 mg, at least 90 mg, at least 100 mg, at least 200 mg, at least 250 mg, at least 300 mg, at least 400 mg, at least 500 mg, at least 600 mg, at least 700 mg, at least 750 mg, at least 800 mg, at least 900 mg, or at least 1000 mg/kg.

In some embodiments, the method can include administering sufficient active agent to provide a maximum daily dose (mg/day) of no more than 8000 mg such as, for example, no more than 5000 mg, no more than 3150 mg, no more than 2000 mg, no more than 1600 mg, no more than 1000 mg/kg, no more than 900 mg, no more than 800 mg, no more than 700 mg, no more than 600 mg, no more than 500 mg, no more than 400 mg, no more than 300 mg, no more than 200 mg, no more than 195 mg, no more than 150 mg, no more than 100 mg, no more than 90 mg, no more than 80 mg, no more than 75 mg, no more than 70 mg, no more than 65 mg, no more than 60 mg, no more than 55 mg, no more than 50 mg, no more than 45 mg, no more than 40 mg, no more than 35 mg, no more than 30 mg, no more than 25 mg, no more than 20 mg, no more than 15 mg, or no more than 10 mg, no more than 3.6 mg, no more than 2 mg, no more than 1.5 mg, no more than 0.01 mg, or no more than 0.004 mg.

In some embodiments, the method can include administering sufficient active agent to provide a daily dose (mg/day) that falls within a range having endpoints defined by any minimum dose listed above and any maximum dose listed above that is greater than the minimum dose. For example, the method can include administering sufficient active agent to provide a dose of from 50 mg to 5000 mg, from 250 mg to 2000 mg, from 500 mg to 1000 mg, from 250 mg to 8000 mg, from 500 mg to 3150 mg, from 750 mg to 1600 mg, from 0.5 mg to 300 mg, from 10 mg to 60 mg, from 20 mg to 40 mg, from 15 mg to 2000 mg, from 30 mg to 300 mg, from 45 mg to 150 mg, from 20 mg to 195 mg, from 33 mg to 100 mg, from 40 mg to 60 mg, from 0.001 mg to 2 mg, from 0.0012 mg to 0.01 mg, or from 0.0024 mg to 0.004 mg.

In certain embodiments, the method can include administering sufficient active agent to provide a daily dose (mg/day) equal in value to any minimum daily dose or any maximum daily does listed above. Thus, for example, the method can include administering sufficient active agent to provide a daily dose (mg/day) of 0.5 mg, 1.5 mg, 3.6 mg, 10 mg, 15 mg, 40 mg, 60 mg, 100 mg, 150 mg, 250 mg, 300 mg, 500 mg, 750 mg, 1600 mg, 2000 mg, etc.

Accordingly, in exemplary embodiments in which the active agent is magnesium or a magnesium salt, the method can include administering sufficient active agent to provide a dose of from 50 mg/day to 5000 mg/day such as, for example, from 250 mg/day to 2000 mg/day. In certain embodiments, the method can include administering sufficient active agent to provide a dose of from 500 mg/day to 1000 mg/day.

In exemplary embodiments in which the active agent is bismuth or a bismuth salt, the method can include administering sufficient active agent to provide a dose of from 250 mg/day to 8000 mg/day such as, for example, from 500 mg/day to 3150 mg/day. In certain embodiments, the method can include administering sufficient active agent to provide a dose of from 750 mg/day to 1600 mg/day.

In exemplary embodiments in which the active agent is molybdenum, molybdate, or a salt of either, the method can include administering sufficient active agent to provide a dose of from 0.5 mg/kg/day to 300 mg/kg/day such as, for example, from 10 mg/kg/day to 60 mg/kg/day. In certain embodiments, the method can include administering sufficient active agent to provide a dose of from 20 mg/kg/day to 40 mg/kg/day.

In exemplary embodiments in which the active agent is zinc or a zinc salt, the method can include administering sufficient active agent to provide a dose of from 15 mg/day to 2000 mg/day such as, for example, from 30 mg/day to 300 mg/day. In certain embodiments, the method can include administering sufficient active agent to provide a dose of from 45 mg/day to 150 mg/day.

In exemplary embodiments in which the active agent is elemental iron or a salt thereof, the method can include administering sufficient active agent to provide a dose of from 20 mg/kg/day to 195 mg/kg/day such as, for example, from 33 mg/kg/day to 99 mg/kg/day. In certain embodiments, the method can include administering sufficient active agent to provide a dose of from 40 mg/kg/day to 60 mg/kg/day.

In exemplary embodiments in which the active agent is nickel or a nickel salt, the method can include administering sufficient active agent to provide a dose of from 0.001 mg/kg/day to 2 mg/kg/day such as, for example, from 0.0012 mg/kg/day to 0.01 mg/kg/day. In certain embodiments, the method can include administering sufficient active agent to provide a dose of from 0.0024 mg/kg/day to 0.004 mg/kg/day.

In exemplary embodiments in which the active agent is nitrate or a nitrate salt, the method can include administering sufficient active agent to provide a dose of from 0.5 mg/kg/day to 3.6 mg/kg day such as, for example, from 1 mg/kg/day to 2.0 mg/kg/day. In certain embodiments, the method can include administering sufficient active agent to provide a dose of 1.5 mg/kg/day.

In exemplary embodiments in which the active agent is cobinamide or a cobinamide salt, the method can include administering sufficient active agent to provide a dose of from 2 mg/kg/day to 5000 mg/kg/day such as, for example, from 10 mg/kg/day to 50 mg/kg/day. In certain embodiments, the method can include administering sufficient active agent to provide a dose of from 15 mg/kg/day to 25 mg/kg/day.

A single dose may be administered all at once, continuously for a prescribed period of time, or in multiple discrete administrations. When multiple administrations are used, the amount of each administration may be the same or different, For example, a dose of 1 mg/day may be administered as a single administration of 1 mg, continuously over 24 hours, as two 0.5 mg administrations, or as a first administration of 0.75 mg followed by a second administration of 0.25 mg. When multiple administrations are used to deliver a single dose, the interval between administrations may be the same or different.

In some embodiments, the active agent may be administered, for example, from a single dose to multiple doses per week, although in some embodiments the method can involve a course of treatment that includes administering doses of the active agent at a frequency outside this range. When a course of treatment involves administering multiple within a certain period, the amount of each dose may be the same or different. For example, a course of treatment can include a loading dose initial dose, followed by a maintenance dose that is lower than the loading dose. Also, when multiple doses are used within a certain period, the interval between doses may be the same or be different.

In some embodiments, the active agent may be administered at least once per day such as, for example, once per day (QD), twice per day (BID), three times per day (TID), or four times per day QID). In certain embodiments, the active agent may be administered twice per day (BID). In some embodiments, the method can include administering active agent at a dosage up to three hours before a meal, with a meal, or up to three hours after a meal.

In some embodiments, the method can include administering active agent at a dosage and frequency listed above for a minimum period of at least three days such as, for example, at least five days, at least seven days, at least 10 days, at least 14 days, or at least 17 days. In some embodiments, the method can include administering active agent at a dosage and frequency listed above for a maximum period of no more than 30 days such as, for example, no more than 28 days, no more than 24 days, no more than 21 days, no more than 17 days, no more than 14 days, or no more than 10 days. In some embodiments, the method can include administering active agent at a dosage and frequency listed above for a period that falls within a range having endpoints defined by any minimum period listed above and any maximum period listed above that is great than the minimum period. Thus, in certain embodiments, the duration of treatment can be from three to 21 days. In some of these embodiments, the duration of treatment can be 10-17 days such as, for example, 14 days. In some embodiment, the method can involving continuing treatment for as long as symptoms and/or clinical signs persist can be repeated if symptoms or clinical sign recur.

Treating a condition can be prophylactic or, alternatively, can be initiated after the subject exhibits one or more symptoms or clinical signs of the condition. Treatment that is prophylactic—e.g., initiated before a subject manifests a symptom or clinical sign of the condition such as, for example, while an infection remains subclinical—is referred to herein as treatment of a subject that is “at risk” of having the condition. As used herein, the term “at risk” refers to a subject that may or may not actually possess the described risk. Thus, for example, a subject “at risk” of a condition is a subject possessing one or more risk factors associated with the condition such as, for example, genetic predisposition, ancestry, age, sex, geographical location, lifestyle, or medical history.

Accordingly, a composition can be administered before, during, or after the subject first exhibits a symptom or clinical sign of the condition. Treatment initiated before the subject first exhibits a symptom or clinical sign associated with the condition may result in decreasing the likelihood that the subject experiences clinical evidence of the condition compared to a subject to which the composition is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

### EXAMPLES

### Example 1

Rats were fed a high fat diet (HFD) to induce dysbiosis and fatty liver disease as previously described (de La Srre et al., 2010, Am J Physiol—Gastrointest Liver Physiol 299:G440-448; Hamilton et al., 2015, Am J Physiol—Gastrointest Liver Physiol 308:G840-851; Etxeberria et al., 2015, J Nutr Biochem 26:651-660; Carmiel-Haggai M., 2004, FASEB J 19:136-138). Responses in control group rats (fed standard chow) were compared to responses in three experimental groups: HFD diet, HFD+BSS, and normal chow+BSS. After eight weeks, all rats returned to normal chow, but the BSS groups continued to receive BSS in the normal chow. After 10 weeks, systemic blood was collected for measurements of liver function tests (AST, ALT, Tbili, Alb) before further experimentation. After blood collection, rats underwent surgery to measure porta vein flow and systemic venous blood pressure before and after infusion of small intestine (jejunum) sodium hydrosulfide (NaHS), a hydrogen sulfide donor. Portal vein flow and systemic venous pressure were recorded at baseline and throughout infusion periods. Liver function tests were reported as mean+/−95% confidence interval and compared using 2-way RM-ANOVA. Blood flow data are reported as mean+/−SEM and were compared by 2-way RM-ANOVA.

### Example 2

Rats were divided into three groups: Magnsium Oxide (MgO) diet, 500 mg MgO per 100 g chow, with NaHS infusion into small intestine (jejunim); standard diet with NaHS infusion into jejunum; and standard diet with control (vehicle) infusion into jejunim. Diets were administered for three days total. At the end of diet treatment, rats underwent surgery to measure porta vein flow before and after infusion of small intestine (jejunum) sodium hydrosulfide (NaHS), a hydrogen sulfide donor. Portal vein blood flow was recorded at baseline for 10 minutes and throughout 20-minute infusion periods. Data reported as percent change from baseline; infusion started at minute 1 of data shown. Blood flow data are reported as mean percent baseline change+/−SEM and were compared by 2-way RM-ANOVA. Statistical significant difference noted at minutes 20 between MgO diet group and other two groups.

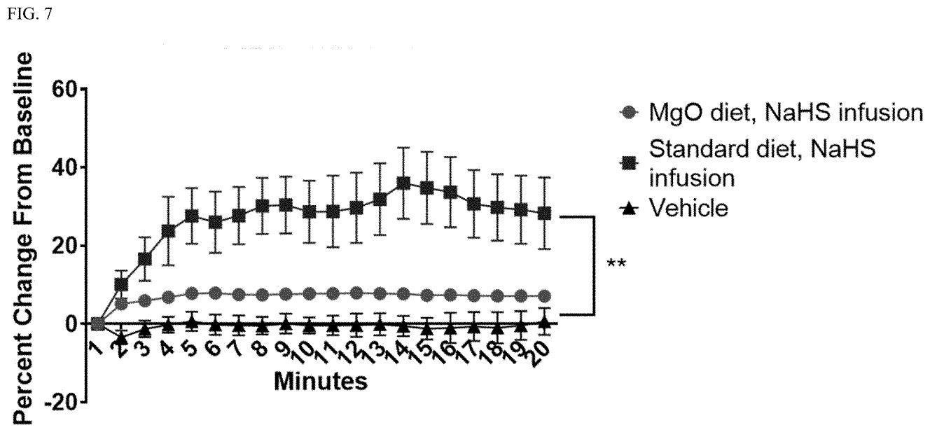

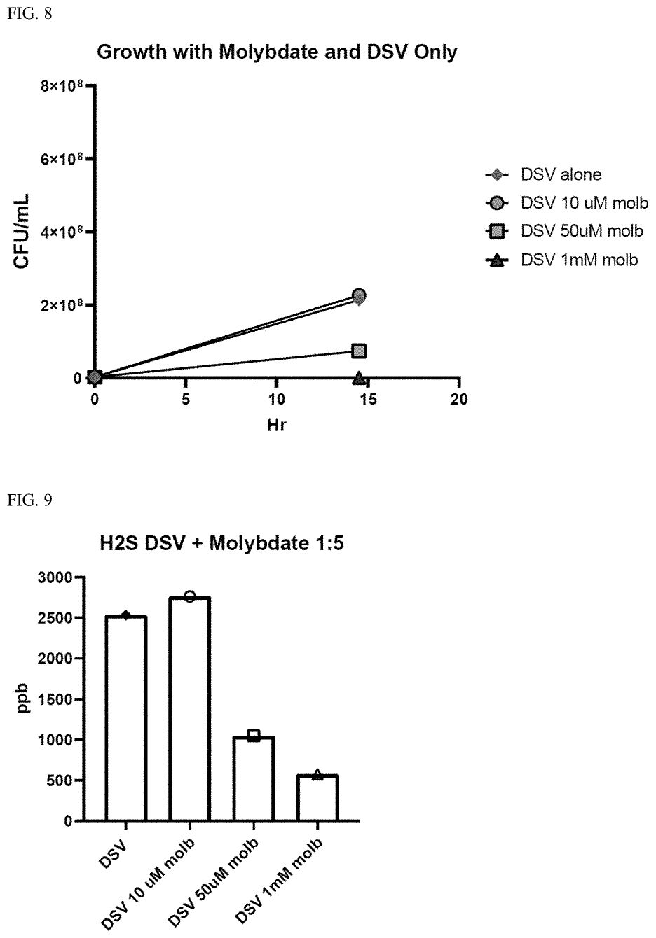

Results are shown in FIG. 7.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A pharmaceutical composition comprising:
a compound that reduces H₂S in the gut of a subject; and
a pharmaceutically acceptable carrier.

2. The pharmaceutical composition of claim 1, wherein the compound that reduces H₂S in the gut of a subject comprises magnesium or a salt thereof, bismuth or a salt thereof, molybdenum or a salt thereof, zinc or a salt thereof, iron or a salt thereof, nickel or a salt thereof, nitrate or a salt thereof, cobinamide or a salt thereof, a heme protein, an antimicrobial, an antibiotic, a prebiotic, a probiotic, or a synbiotic.

3. The pharmaceutical composition of claim 2, wherein the magnesium salt is magnesium hydroxide.

4. The pharmaceutical composition of claim 2, wherein the bismuth salt is bismuth subsalicylate.

5. The pharmaceutical composition of claim 2, wherein the zinc salt is zinc oxide.

6. A method of detecting H₂S-mediated hyperdynamic circulation, or a consequence thereof, in a subject having, or at risk of having, H₂S-mediated hyperdynamic circulation, the method comprising:
obtaining a biological sample from the subject;
measuring H₂S in the sample; and
identifying the subject as having H₂S-mediated hyperdynamic circulation if the H₂S measured in the sample is greater than a predetermined threshold.

7. The method of claim 6, wherein the biological sample comprises plasma or blood.

8. The method of claim 7, wherein the predetermined threshold is a plasma H₂S concentration of 45 μM.

9. The method of claim 7, wherein the predetermined threshold is a baseline plasma H₂S concentration measured from a previous sample from the subject.

10. The method of claim 6, wherein the biological sample comprises a breath.

11. The method of claim 10, wherein the predetermined threshold is a baseline breath H₂S concentration measured from a previous sample from the subject.

12. A method of detecting H₂S-mediated hyperdynamic circulation, or a consequence thereof, in a subject having, or at risk of having, H₂S-mediated hyperdynamic circulation, the method comprising:
obtaining a biological sample from the subject;
measuring thiosulfate in the sample; and
identifying the subject as having H₂S-mediated hyperdynamic circulation if the thiosulfate measured in the sample is greater than a predetermined threshold.

13. The method of claim 12, wherein the biological sample comprises plasma or blood.

14. The method of claim 13, wherein the predetermined threshold is a plasma thiosulfate concentration of 1.13 mg/dl.

15. The method of claim 13, wherein the predetermined threshold is a baseline plasma thiosulfate concentration measured from a previous sample from the subject.

16. The method of claim 12, wherein the biological sample comprises urine.

17. The method of claim 16, wherein the predetermined threshold is a urine thiosulfate concentration of 0.28 mg/dl.

18. The method of claim 16, wherein the predetermined threshold is a baseline urine thiosulfate concentration measured from a previous sample from the subject.

19. A method of treating a subject having, or at risk of having, H₂S-mediated hyperdynamic circulation or a consequence of H₂S-mediated hyperdynamic circulation, the method comprising:
administering to the subject a composition comprising a compound that reduces H₂S in the gut of the subject in an amount effective to treat H₂S-mediated hyperdynamic circulation or the consequence of H₂S-mediated hyperdynamic circulation.

20. The method of claim 19, wherein the compound that reduces H₂S in the gut of a subject comprises magnesium or a salt thereof, bismuth or a salt thereof, molybdenum or a salt thereof, zinc or a salt thereof, iron or a salt thereof, nickel or a salt thereof, nitrate or a salt thereof, cobinamide or a salt thereof, a heme protein, an antimicrobial, an antibiotic, a prebiotic, a probiotic, or a synbiotic.

21. The method of claim 19, wherein the consequence of H₂S-mediated hyperdynamic circulation comprises portal hypertension, varices formation, variceal bleeding, portal hypertensive gastropathy, gastric antral vascular ectasia, portal hypertensive enteropathy, portal hypertensive colonopathy, liver failure, hepatic encephalopathy, coma, variceal bleeding, hypervolemic hyponatremia, electrolyte imbalances, renal failure, sodium retention, fluid retention, tissue edema, portopulmonary syndrome, hepatopulmonary syndrome, pulmonary hypertension, jaundice, splenomegaly, portosystemic anastomosis formation, impaired hepatic detoxification, bacterial translocation, liver failure, fibrosis formation cirrhosis and its complications, brain swelling, hepatorenal syndrome, ascites, spontaneous bacterial peritonitis, pleural effusion, hypoxemia, hypoxia, hepato-pulmonary syndrome, porto-pulmonary syndrome, hepatic cardiac syndrome, cirrhotic cardiomyopathy, or heart failure.

22. The method of claim 19, wherein the H₂S-mediated hyperdynamic circulation is related to obesity, fatty liver disease, metabolic syndrome, alcoholic liver disease, steatosis, steatohepatitis, hepatitis, cirrhosis, other acute or chronic liver disease, heart failure, respiratory failure, or kidney failure.

23. The method of claim 19, wherein the treatment is prophylactic.

24. The method of claim 23, wherein the effective amount is an amount effective to:
decrease the likelihood that the subject experiences clinical evidence of the condition compared to a subject to whom the composition is not administered;
decreasing the severity of a symptom of the condition compared to a subject to whom the composition is not administered;
decreasing the severity of a clinical signs of the condition compared to a subject to whom the composition is not administered; or
resolve the condition.

25. The method of claim 24, wherein the symptom or clinical sign comprises hematemesis, coffee-ground emesis, hematochezia, passing blood from the rectum, passing melena from the rectum, easy bruising, nausea, vomiting, early satiety, gastroesophageal reflux, dysphagia, anorexia, bloating, excessive gas, excessive flatus, jaundice, confusion, altered mental status, difficulty with concentration, impaired memory, insomnia, asterixis, fogginess of head, edema, abdominal swelling, abdominal pain, difficulty with breathing, shortness of breath, fatigue, change in weight, chest pain, palpitations of the heart, decreased urine output, leg edema or swelling, or anasarca.

26. The method of claim 19, wherein the treatment is therapeutic.

27. The method of claim 26, wherein the effective amount is an amount effective to:
decrease the severity of a symptom of the condition compared to a subject to which the composition is not administered;
decrease the severity of a clinical sign of the condition compared to a subject to which the composition is not administered; or
resolve the condition.

28. The method of claim 27, wherein the symptom or clinical sign comprises hematemesis, coffee-ground emesis, hematochezia, passing blood from the rectum, passing melena from the rectum, easy bruising, nausea, vomiting, early satiety, gastroesophageal reflux, dysphagia, anorexia, bloating, excessive gas, excessive flatus, jaundice, confusion, altered mental status, difficulty with concentration, impaired memory, insomnia, asterixis, fogginess of head, edema, abdominal swelling, abdominal pain, difficulty with breathing, shortness of breath, fatigue, change in weight, chest pain, palpitations of the heart, decreased urine output, leg edema or swelling, or anasarca.
